(19) United States
(12) Patent Application Publication
(10) Pub. No.: US 2002/0134386 A1
(43) Pub. Date: Sep. 26, 2002

(54) Regulator for underwater breathing apparatus

(76) Inventor: Giovanni Garofalo, Rapallo (IT)

Correspondence Address: LARSON & TAYLOR, PLC, 1199 NORTH FAIRFAX STREET, SUITE 900, ALEXANDRIA, VA, 22314, US

(21) Appl. No.: 10/095,438

(22) Filed: Mar. 13, 2002

(30) Foreign Application Priority Data
Mar. 23, 2001 (IT) GE2001 A 000028

(51) Int. Cl. A62B 18/10; A62B 7/04; A61M 16/00; F16K 31/26

(52) U.S. Cl. 128/204.26; 128/201.28

(57) ABSTRACT

Regulator for underwater breathing apparatus including: a box-like body having a mouthpiece; an air inlet duct connected to said box-like body through an inlet valve; one or more exhaust ducts connected to said box-like body; a flexible diaphragm frontally positioned on the opposite side as to said mouthpiece and co-operating with an opening or closing lever of said inlet valve, said regulator includes means manually workable from the outside and movable from an active position, where they limit the moving of the said lever to the inside of the said box-like body, to an inactive position where they allow said lever a complete transfer movement to the inside of said box-like body.

### BACKGROUND AND SUMMARY OF THE INVENTION

The present invention refers to the regulators for underwater breathing apparatus.

As known, said regulators include a flexible diaphragm which, through the difference in pressure between the chamber of the regulator and the outer environment which is generated whenever the diver is inhaling, operates a lever coupled in well known manner to the valve controlling of the air flow from the compressed air source. It happens that during the phase of entry into water of the diver, for instance with a backward dive from a boat, it is possible that said diaphragm be subjected to an excessive flexion due to the sudden outer pressure increase caused by the impact with the water, which in turn causes the excessive movement of the lever coupled to the valve controlling the air flow, giving rise to unwanted self-delivery effects.

It is therefore the main object of the present invention to obviate to the drawbacks of the known regulators by providing the regulators with a manually operable device which which may be operated from a first operational position in which it is capable to prevent, when the diver enters the water, the excessive shifting of the lever associated to the valve controlling the air flow and which, when the diver is under water, may be shifted to a second operational position in which it allows the said lever to perform its normal control function of the compressed air inlet duct.

According to the present invention said object is obtained by providing a regulator for underwater breathing apparatus of the kind comprising a box-like body having a first and end a second end; a chamber in said body, said chamber containing a regulating device for regulating the flow of air; a mouthpiece tube connected to the first end of the said body and carrying a mouthpiece; an air inlet hose connected to the said body by means of an air inlet valve, the valve being controlled by the regulating device; a diaphragm for actuating the regulating device when a user inhales; at least on exhaust hose connected to the body; and means manually operable from the outside and movable from a first position, in which they limit the movement of the said regulating device, to a second position in which they allow the unlimited movement of said regulating device.

### BRIEF DESCRIPTION OF THE DRAWINGS

Further objects and advantages of the present invention will be better understood from the following description, made with reference to the enclosed drawing, in which:

FIG. 1 is a partially sectioned top plan view of a preferred embodiment of a regulators for underwater breathing apparatus according to the present invention.

### DESCRIPTION OF THE PREFERRED EMBODIMENT OF THE INVENTION

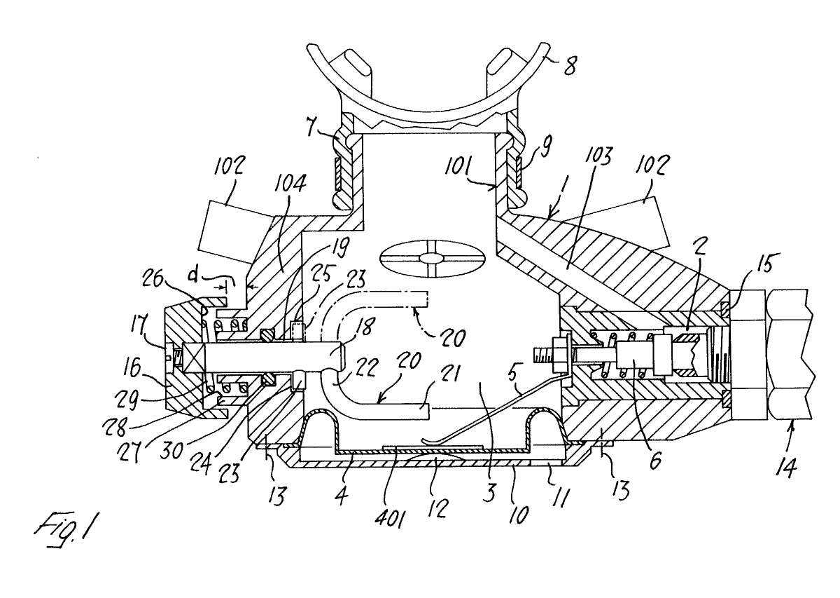

In FIG. 1 a regulator for underwater breathing apparatus is shown comprising a box-like body 1 housing a device (3, 4, 5, 6) for the regulation of the air flow coming from an inlet duct 2. Said regulating device essentially consists of a chamber 3 having a flexible diaphragm 4 located at the end of the box-like body 1 opposite to the end with the mouthpiece 8; said flexible diaphragm 4, for the difference of pressure which creates when the diver inhales, operates the lever 5 coupled to the air flow adjustment valve 6, so that said valve 6 opens the inlet duct 2. Said duct 2 is connected through a joint 14 to a compressed air source, as for instance compressed air bottles (not shown), being for said connection provided sealing means 15. The box-like body 1 includes moreover an integral pipe 101 connected with a joint 7 of the mouthpiece 8, said joint 7 being advantageously made of elastomeric material and provided with a sealing ring 9 for the regulator. The box-like body 1 includes moreover two exhaust ducts 102, positioned sideways as to the diver's mouthpiece 8, and a by-pass pipe 103 integral with the box-like body 1. The regulator includes moreover an hard cap 10 in connection with the flexible diaphragm 4. Said cap 10 is provided with an opening 11 for the communication of saiddiaphragm 4 with the surrounding water. In the present embodiment said cap 10 is fastened through pins 13 to the box-like body 1 and the flexible diaphragm 4, being inserted at its ends between the box-like body 1 and the cap 10, assures an effective sealing of the breathing device. Moreover it is present, in the known way, a button 12 for the discharging and control of the regulator.

On a side wall 104 of the box-like body 1 near the one where the diaphragm 4 is housed in, in this case it is the wall opposite to the compressed air inlet duct 2, it is provided a device limiting the movement of the lever 5 to the inside of the chamber 3. Said device includes a knob 16 connected through a screw 17 to an end of an spindle 18 housed within a through hole 19 formed on the side wall 104 of the regulator. The other end of the spindle 18 extends within the regulator chamber 3 and to said end a substantially “L” shaped element formed by a base 21 parallel and misaligned as to the spindle 18 and by a shank 22 orthogonal to said spindle 18 is secured. Near said “L” shaped element 20, in a direction perpendicular to the axis of the spindle 18, a pin 23 is secured to said spindle 18. Said pin 23 may be accomodated in one of two relative diametrically opposed seats 24 and 25 formed on the inner side wall 104 of the regulator. As mentioned, said spindle 18 has an end extending outside of the regulator to which it is connected a knob 16. Said knob 16 is internally provided with a cylindrical seat 26 coupled to a cylindrical pin 27 projecting outward from the side wall 104. Said cylindrical pin 27 has an axial annular groove 28 in which it is inserted one end of a compressed spring 29, whose other end is fastened in the bottom wall of the cylindrical seat 26 of the knob 16. Moreover, in order to guarantee the watertight sealing of the above described limiting device of the movement of the lever 5, it is provided a sealing ring 30 inserted between the spindle 18 and the inner wall of the through hole 19.

The operation of said limiting device is as follows: normally the “L” shaped element 20 is in an inactive position, it is to say the position shown with the dotted line in figure, with the pin 23 inserted within the upper seat 25, in said position the lever 5 is free to move to the inside of the chamber 3 and then co-operate with the valve 6 for the opening of the air inlet duct 2; when the diver is going to dive into the water, to avoid that the lever 5 performs said shifting, fact which would cause unwanted self-supplying effects, he has to make the spindle 18 rotate so that the “L” shaped element 20 presents with the base 21 next to the end of the arm 5, it is to say in an active position; the diver then takes the knob 16 and pushes the spindle 18 to the inside, against the work of the spring 29 actually compressed, so to make it run the distance d, necessary to release the pin 23 from the seat 25; at this point, by rotating the knob 16 to the lower part of the figure, it is possible to lead the pin 23 next to the seat 24, where said pin will be withdrawn thanks to the return work of the spring 29, which, then, will be slackened back. In this way the movement of the lever 5 to the inside of the chamber is limited from the contact of the end of said lever 5 against the base 21 of the “L” shaped element correctly positioned in active position. When the diver has entered the water, to restore the normal functioning of the regulator, he has just to perform with the knob 16 the operation contrary to the just described one, leading the “L” shaped element 20 back to the inactive position shown by the dotted line.

I claim

1. Regulator for underwater breathing apparatus of the kind comprising a box-like body having a first and end a second end; a chamber in said body, said chamber containing a regulating device for regulating the flow of air; a mouthpiece tube connected to the first end of said body and carrying a mouthpiece; an air inlet hose connected to said body by means of an air inlet valve, the valve being controlled by the regulating device; a diaphragm for actuating the regulating device when a user inhales; at least on exhaust hose connected to the body; and means manually operable from the outside and movable from a first position, in which they limit the movement of the said regulating device, to a second position in which they allow the unlimited movement of said regulating device.

2. The regulator according to claim 1, in which the said manually operable means comprises a spindle bearing at one end an L shaped arm and provided at its other end with a suitable handgrip element, and in which in said box-like body is provided, near the side where the said diaphragm is positioned, with a through hole through which the said spindle of said manually operable means is rotatably and axially shiftably inserted, with the said handgrip element at the exterior of the said box like member and with the said L shaped element housed in the said chamber of the said box like member.

3. The regulator according to claim 2, in which said regulating device is in the form of a lever cooperating with the said diaphragm, and the said L shaped element of the said manually operable means comprises a base element substantially parallel to said spindle and a shaft substantially orthogonal to said spindle, said base element being able to co-operate with the said lever so as to limit its movement in the said active position of said L shaped element.

4. The regulator according to claim 3, in which said spindle is formed with one pin extending at right angles with respect to said spindle axis and in which in the inner side wall of the said box-like body are formed at least two seats which are apt to accomodate said pin, being one said seats fit to accomodate the said pin in the active position of the said L shaped element and the other of said seats being fit to accomodate said pin in the inactive position of said L shaped element.

5. The regulator according to claim 4,further comprising spring means for returning said pin in one of said seats.

6. The regulator according to claim 3, in which a knob is fastened to the end of said spindle extending outside of the said box-like body, by means of which said spindle may be axially shifted and/or rotated in said through hole.

7. The regulator according to claim 6, in which the said knob is provided, on the side facing the regulator side wall, with a cylindrical seat in which a cylindrical pin projecting out of said side wall is inserted, the said cylindrical pin being provided with at least one groove in which one end of said spring means is inserted, whose other end is fastened to the bottom wall of said cylindrical seat of said knob.

8. The regulator according to claim 2, in which said knob is fastened to said spindle through removable means.

9. The regulator according to claim 2,in which between the said spindle and the inner wall of the said through hole at least one sealing ring is provided.
